(19) United States
(12) Patent Application Publication
(10) Pub. No.: US 2025/0057785 A1
(43) Pub. Date: Feb. 20, 2025

(54) APPLICATION OF 10'(Z), 13'(E), 15'(E)-HEPTADECATRIENYL HYDROQUINONE (HQ17(3)) IN TREATING CORONAVIRUS INFECTION

(71) Applicant: National Atomic Research Institute, Taoyuan (TW)

(72) Inventors: MEI-HUI WANG, Taoyuan (TW); Kun-Liang LIN, Taoyuan (TW); Hung-Wen YU, Taoyuan (TW); Sui-Yuan CHANG, Taoyuan (TW); Chung-Yi HU, Taoyuan (TW); Shwu-Bin AU LIN, Taoyuan (TW)

(21) Appl. No.: 18/385,444

(22) Filed: Oct. 31, 2023

(30) Foreign Application Priority Data
Aug. 15, 2023 (TW) 112130678

(51) Int. Cl. A61K 31/05; A61P 31/14

(52) U.S. Cl. CPC A61K 31/05; A61P 31/14

(57) ABSTRACT

The present disclosure relates to use of 10′(Z), 13′(E), 15′(E)-Heptadecatrienyl hydroquinone compound (hereafter “HQ17(3)”) represented by the following Formula (12), a pharmaceutically acceptable salt, and/or a solvate and/or a hydrate thereof, and a pharmaceutical composition comprising the above compound, in treating coronavirus infection and diseases caused by the infection, especially SARS-COV-2 infection.

### CROSS REFERENCE TO RELATED APPLICATION

All related applications are incorporated by reference. The present application is based on Taiwan Application Serial Number 112130678, filed on Aug. 15, 2023. The disclosure of which is hereby incorporated by reference herein in its entirety.

### TECHNICAL FIELD

The present disclosure relates to use of 10′(Z), 13′(E), 15′(E)-Heptadecatrienyl hydroquinone compound (hereafter “HQ17(3)”) represented by the following Formula (12), a pharmaceutically acceptable salt, and/or a solvate and/or a hydrate thereof, and a pharmaceutical composition comprising the above compound, in treating coronavirus infections and diseases caused by the infections, especially SARS-COV-2 infection.

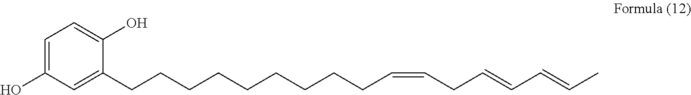 [SMILES: C/C=C/C=C/C/C=C\CCCCCCCCCc1cc(O)ccc1O]

### BACKGROUND

The global pandemic of coronavirus disease 2019, caused by severe acute respiratory syndrome coronavirus 2 (SARS-COV-2), broke out in December 2019, and has caused hundreds of millions of people infected globally, tens of millions of them were died. The World Health Organization (WHO) finalized the official name of the disease caused by SARS-COV-2 as the COVID-19 (Coronavirus Disease-2019).

The novel coronavirus (SARS-COV-2) belongs to the beta subfamily Coronavirinae of the family Coronaviridae. Coronaviruses can cause diseases in humans and vertebrate animals, and these diseases are mostly zoonotic diseases. Seven coronaviruses are known to infect humans, including: HCoV-229E and HCoV-NL63 of the alpha subfamily, HCoV-HKU1, HCoV-OC43, and severe acute respiratory syndrome coronavirus (SARS-COV) of the beta subfamily, Middle East Respiratory Syndrome Coronavirus (MERS-COV) and the newly discovered novel coronavirus (SARS-COV-2).

No specific and effective treatment or cure for all of the above-mentioned coronavirus infections has been reported, thus far. Actually, the supportive care is the most commonly used treatment in the infected cases to relieve patients' symptoms of the infection. Several drugs have been reported to be effective in inhibiting the novel coronavirus (SARS-COV-2), however, most of them are restricted by the severe side effects while in treating of the novel coronavirus (SARS-COV-2) infection.

For example, Remdesivir, an antiviral drug known as a nucleotide analogue, can inhibit the replication of coronavirus by competing with viral RNA in binding to viral RNA polymerase in theory. However, according to large-scale clinical data acquired during the pandemic of COVID-19, the administration of Remdesivir to severe cases who were intubated or equipped with Extracorporeal Membrane Oxygenation (ECMO) revealed no effective reduction of mortality or prognosis significantly. Instead, in some cases, the mortality rate were even observed to be slightly increased after the treatment of Remdesivir.

The increase in mortality rate is believed to be due to the side effects caused by Remdesivir. According to the Remdesivir Emergency Use Authorization instructions, Remdesivir may cause several side effects such as hypotension, nausea, vomiting, sweating and shaking, and liver inflammation.

An additional example, Tocilizumab, a monoclonal antibody, is an IL-6 (Interleukin-6) pathway inhibitor. According to the information provided by the Department of Disease Control and Prevention, Ministry of Health and Welfare, Taiwan, the research results revealed that COVID-19 patients treated with IL-6 pathway inhibitors were observed to have significantly reduced inflammation level, however, only around 30% were clinically improved. Several side effects may be caused in the treatment including upper respiratory tract infection, nasopharyngitis, headache, hypertension, etc.

Another example, Hydroxychloroquine, which is known to be used to treat malaria, is also used to treat COVID-19 currently, despite the mechanism of that is still unknown. Side effects caused by Hydroxychloroquine may include gastrointestinal upset, skin rash, pigmentation, tinnitus, asthenia, abnormal vision etc. There is lack of effective evidence showing that Hydroxychloroquine can prevent or cure COVID-19.

An additional example, the use of the antibiotic Azithromycin, an antimycoplasma drug, is known as one of the auxiliary therapies for COVID-19. Common side effects of this drug include palpitations, diarrhea, abdominal pain, etc.

According to the information on the website of Taiwan Centers for Disease Control, the Ministry of Health and Welfare, approximately 14% of SARS-COV-2 infected patients appeared severe symptoms and were requiring hospitalization and oxygen therapy. Most of the death cases of the infected patients were observed to have underlying health conditions. Therefore, for the development of therapeutic drugs for the novel coronavirus infection, side effects of drugs are crucial factors in considering drug safety.

Several researches on 10′(Z), 13′(E), 15′(E)-Heptadecatrienyl hydroquinone compound (HQ17(3)) as an anti-cancer drug have been reported. The results indicated that, without treatment, blood cancer HL-60 cells can replicate and grow to 2.5 times in 48 hours, however, when 5 uM HQ17(3) was added to blood cancer HL-60 cells and cultured for 48 hours, the replication of blood cancer HL-60 cells was stopped, showing an effective inhibitory effect. Relatively, the peripheral white blood cells was not expected to replicate. There was no proliferation been observed even after 48 hours of culturing. Addition of 5 uM HQ17(3) to peripheral white blood cells showed no effect on peripheral white blood cells, even when the dosage was as high as 50 uM HQ17(3), no toxicity on peripheral white blood cells was observed. In addition, studies indicated that no toxicity was observed even after intraperitoneal injection of 1 mg/kg HQ17(3) into F344 rats daily even for 28 days (Toxicology and Applied Pharmacology 227 (2008) 331-338).

The 10′(Z), 13′(E), 15′(E)-Heptadecatrienyl hydroquinone compound (HQ17(3)) is known can effectively inhibit the growth of cancer cells, but does not have any effect on non-replicating peripheral blood cells. The novel coronavirus (SARS-COV-2) is known to mainly invade the lungs, and because the alveolar cells do not replicate, thus, HQ17(3) is expected theoretically to only inhibit replicating coronaviruses (such as SARS-COV-2) but not to affect the non-replication cells. Hence, HQ17(3) can potentially be used as a therapeutic drug which can effectively inhibit the replication of the novel coronavirus (SARS-COV-2).

HQ17(3) can be extracted from Taiwanese sumac (Rhus succedanea), however, the yield of HQ17(3) extracted from Taiwanese sumac is very limited. Currently, the most of medical researches of HQ17(3) are restricted to in vitro cell experiments and basic animal experiments. The limited yield of HQ17(3) makes the translational research in the pharmaceutical industry even more difficult.

In view of the aforementioned, the inventor of the present disclosure had provided a synthesis method of 10′(Z), 13′(E), 15′(E)-Heptadecatrienyl hydroquinone compound derivatives (TWI718699), which allowed us to obtain HQ17(3) derivatives by chemical synthesis with higher productivity and thus, to promote the development of commercial production processes. In addition, the present disclosure uses the above-mentioned synthesis method, to provide 10′(Z), 13′(E), 15′(E)-Heptadecatrienyl hydroquinone compound (HQ17(3)) as shown in Formula (12), and its pharmaceutically acceptable salts and/or its solvates and/or its hydrates, and pharmaceutical compositions comprising the compound, and the use of the above compounds and the pharmaceutical compositions in preparation of medicament for treating coronavirus infections and the diseases caused by the infections, especially the novel coronavirus (SARS-COV-2) infection.

### SUMMARY

To provide a reader with a basic understanding of the present disclosure, the summary provides a brief description of the disclosure. The summary is not a complete description of the disclosure, and is not intended to limit the technical features or the scope of this application.

In order to find an effective and safe drug for the treatment of coronavirus infections, especially the novel coronavirus infection, the instant disclosure demonstrates and provides the 10′(Z), 13′(E), 15′(E)-Heptadecatrienyl hydroquinone compound (HQ17(3)) as shown in Formula (12) having effective inhibition of the replication of SARS-COV-2 coronavirus in the research results, indicating the HQ17(3) can be used in the preparation of a medicament for treating coronavirus infections and the diseases caused by the infections, especially the novel coronavirus (SARS-COV-2) infection.

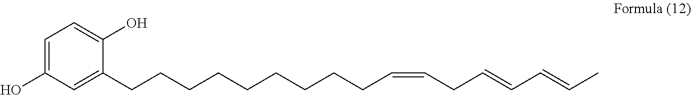 [SMILES: C/C=C/C=C/C/C=C\CCCCCCCCCc1cc(O)ccc1O]

The present disclosure relates to a 10′(Z), 13′(E), 15′(E)-Heptadecatrienyl hydroquinone compound (HQ17(3)) represented by Formula (12), and a pharmaceutically acceptable salt, and/or a solvate, and/or a hydrate thereof, and a pharmaceutical composition comprising HQ17(3), and the use of the above compounds and the pharmaceutical composition comprising HQ17(3) in preparation of a medicament for treating coronavirus infections and the diseases caused by the infections, especially the novel coronavirus (SARS-COV-2) infection.

In some embodiments of the present disclosure, the pharmaceutically acceptable salt of the HQ17(3) compound shown in Formula (12) of the present disclosure include inorganic salt or organic salt thereof. The present disclosure relates to all forms of the above-mentioned salts, including but not limited to: sodium salt, potassium salt, calcium salt, lithium salt, hydrochloride salt, nitrate salt, phosphate, sulfate, oxalate, acetate, propionate, citrate, benzoate, benzenesulfonate and succinate and so on.

The HQ17(3) compound presented in Formula (12) of the present disclosure can effectively inhibit the replication of coronavirus in cells, especially the replication of SARS-COV-2 coronavirus.

In some embodiment of the present disclosure, the HQ17(3) compound represented by Formula (12) can inhibit the replication of SARS-COV-2 coronavirus in cells by 50% at a concentration of 8.5±0.96 uM.

The present disclosure also relates to the use of HQ17(3) compound represented by Formula (12), a pharmaceutically acceptable salt, and/or a solvate and/or a hydrate thereof in preparation of a medicament for inhibiting the replication of coronavirus in cells, especially in mammalian cells. In some embodiments, the coronavirus in the present disclosure is the novel coronavirus (SARS-COV-2).

The present disclosure also relates to the use of HQ17(3) compound represented by Formula (12), a pharmaceutically acceptable salt, and/or a solvate and/or a hydrate thereof in preparation of a medicament as coronavirus inhibitor. In some embodiments, the coronavirus in the present disclosure is the novel coronavirus (SARS-COV-2).

In some embodiments of the present disclosure, the above-mentioned cells are mammalian cells.

The present disclosure also relates to a pharmaceutical composition, which comprises a therapeutically effective amount of the HQ17(3) compound represented by Formula (12), a pharmaceutically acceptable salt, and/or a solvate, and/or a hydrate, and a pharmaceutically acceptable carrier, auxiliaries or excipient.

The present disclosure also relates to the use of a pharmaceutical composition in preparation of a medicament for treating coronavirus infections and diseases caused by the infections, wherein the pharmaceutical composition comprises the therapeutically effective amount of HQ17(3) compound presented by Formula (12), a pharmaceutical acceptable salt, and/or a solvate and/or a hydrate thereof.

In some embodiments of the present disclosure, the pharmaceutical composition of the present disclosure further comprises a pharmaceutically acceptable carrier, auxiliaries or excipient.

In some embodiments, the pharmaceutical composition of the present disclosure is a solid preparation, an injection, a spray, a liquid pharmaceutical or a composite pharmaceutical.

The present disclosure also relates to use of 10′(Z), 13′(E), 15′(E)-Heptadecatrienyl hydroquinone compound (HQ17(3)) represented by Formula (12), a pharmaceutically acceptable salt, and/or a solvate, and/or a hydrate thereof or a pharmaceutical composition comprising the HQ17(3) compound represented by Formula (12), in preparation of a medicament, wherein the medicament is used to treat a disease or infection in a subject in need thereof caused by coronavirus.

In some embodiments of the present disclosure, the subject in need is a mammal. In some other embodiments, the subject in need is a coronavirus-infected human.

In some embodiments of the present disclosure, the disease caused by the coronavirus infection, especially the SARS coronavirus (SARS-COV) infection or the novel coronavirus (SARS-COV-2) infection, is a respiratory disease, such as: pneumonia, acute respiratory infection, severe acute respiratory syndrome (SARS) and so on.

In some embodiments of the present disclosure, the coronavirus is HCoV-229E alpha coronavirus, HCoV-NL63 alpha coronavirus, HCoV-HKU1 beta coronavirus, HCoV-OC43 beta coronavirus, severe acute respiratory syndrome coronavirus virus (SARS-COV), Middle East Respiratory Syndrome coronavirus (MERS-COV) or the novel coronavirus (SARS-COV-2).

In some embodiments of the present disclosure, the coronavirus is the novel coronavirus (SARS-COV-2).

In the present disclosure, the term “therapeutically effective amount” refers to an amount that is effective in treating an individual in need and thus resulting in the desired therapeutic, ameliorative, inhibitory or preventative effect of the disease or symptom. The therapeutically effective amount may be determined and adjusted basing on physical condition of the individual being treated, the age, weight of individual, severity of the condition, the route of administration, the method of treatment and so on.

The 10′(Z), 13′(E), 15′(E)-Heptadecatrienyl hydroquinone compound (HQ17(3)), represented by Formula (12), used in the present disclosure is obtained by the chemical synthesis method (TWI718699), which was previously proposed by the inventor of this application. The chemical synthesis method used hydroquinone as a starting material to synthesize oxynonyl-acetoxy benzene, and followed by subjecting oxynonyl-acetoxy benzene to a Wittig Reaction with heptadiene-triphenylphosphine to obtain heptadecatrienyl alkyl acetoxybenzene. The obtained heptadecatrienyl alkyl acetoxybenzene was then treated with sodium methoxide under methanol condition to obtain the final pure solid state of heptadecatrienyl hydroquinone. The advantage of using the chemical synthesis method is that using hydroquinone as a starting material is more stable than catechol and the product is non-oily but solid pure substance. Secondly, the deacetylation protection method in the related art (the anticancer drugs based on catechol derivatives) used lithium aluminum hydride (LiAlH4) to deacetylate, but by such method was not able to obtain the final product of heptadecatrienyl hydroquinone. The use of sodium methoxide to achieve deacetylation can result in a high-purity solid product without causing other oxidation effects on the acyl bond, which is better than the lithium aluminum hydride deacetylation.

The present disclosure uses a 10′(Z), 13′(E), 15′(E)-Heptadecatrienyl hydroquinone compound (HQ17(3)) represented by Formula (12), which is obtained by the chemical synthesis method as summarized in follows, including:
(a) subjecting dimethoxybenzene to bromoalkylation and demethylation reactions to generate a compound of Formula (3);

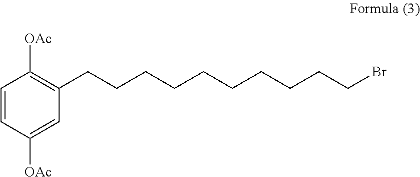 [SMILES: CC(=O)Oc1ccc(OC(C)=O)c(CCCCCCCCCCBr)c1]

(b) subjecting the compound of Formula (3) to iodination and oxidization reactions to generate a compound of Formula (5); and

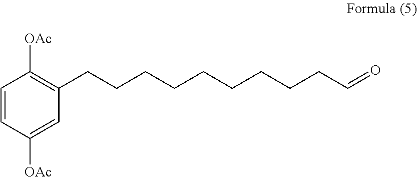 [SMILES: CC(=O)Oc1ccc(OC(C)=O)c(CCCCCCCCCC=O)c1]

(c) subjecting the compound of Formula (5) and heptadien-1-triphenylphosphonium iodide to a Wittig reaction and a deacetylation reaction to obtain hydroquinone derivatives with heptadecatrienyl side chain; for example, a compound of Formula (12).

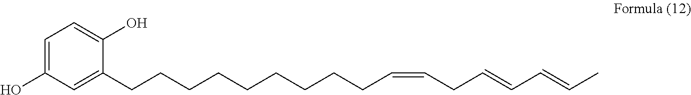 [SMILES: C/C=C/C=C/C/C=C\CCCCCCCCCc1cc(O)ccc1O]

According to an embodiment of the present disclosure, the heptadien-1-triphenylphosphonium iodide in step (c) includes a compound of Formula (10).

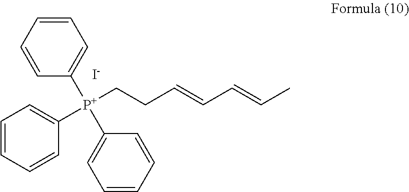 [SMILES: C/C=C/C=C/CC[P+](c1ccccc1)(c1ccccc1)c1ccccc1 ; [I-]]

According to another embodiment of the present disclosure, in step (c), the hydroquinone derivative with heptadecatrienyl side chain is a compound of Formula (11) obtained by subjecting the compound of Formula (5) and the compound of Formula (10) to the Wittig reaction.

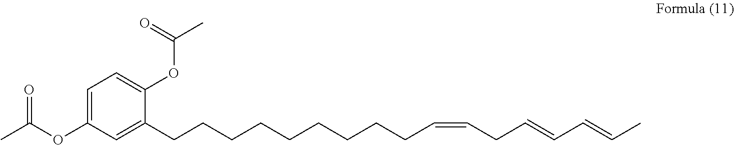 [SMILES: C/C=C/C=C/C/C=C\CCCCCCCCCc1cc(OC(C)=O)ccc1OC(C)=O]

According to another embodiment of the present disclosure, the deacetylation reaction in step (c) is carried out by subjecting the compound of Formula (11) to the deacetylation reaction to obtain a compound of Formula (12).

In addition, in an embodiment of the present disclosure, the deacetylation reaction in step (c) is carried out by reacting the compound of Formula (11) with sodium methoxide in methanol at room temperature.

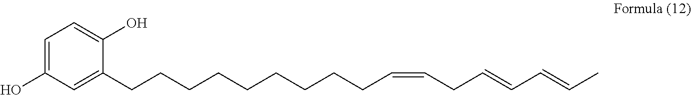 [SMILES: C/C=C/C=C/C/C=C\CCCCCCCCCc1cc(O)ccc1O]

In addition, in an embodiment of the present disclosure, the deacetylation reaction in step (c) is carried out by reacting the compound of Formula (11) with sodium methoxide in methanol at room temperature.

In an embodiment of the present disclosure, the bromoalkylation reaction of dimethoxybenzene in step (a) is carried out by activating dimethoxybenzene with n-butyl lithium and then reacting with 1,10-dibromodecane by one step to obtain a compound of Formula (1),

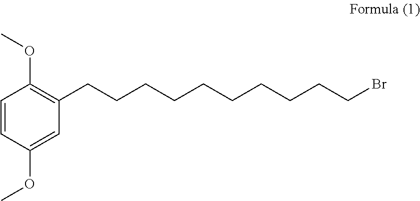 [SMILES: COc1ccc(OC)c(CCCCCCCCCCBr)c1]

then reacting with boron tribromide to demethylate to obtain a compound of Formula (2),

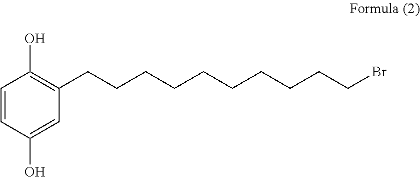 [SMILES: Oc1ccc(O)c(CCCCCCCCCCBr)c1]

and lastly, reacting acetic anhydride with pyridine to obtain the compound of Formula (3).

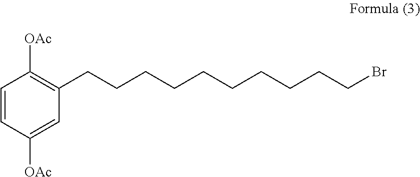 [SMILES: CC(=O)Oc1ccc(OC(C)=O)c(CCCCCCCCCCBr)c1]

According to an implementation method of this application, the iodination reaction in step (b) is carried out by adding sodium iodide to react with and to obtain a compound of Formula (4).

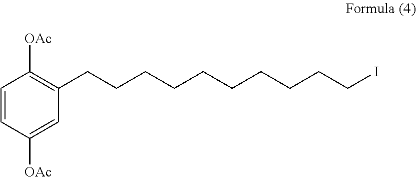 [SMILES: CC(=O)Oc1ccc(OC(C)=O)c(CCCCCCCCCCI)c1]

According to a specific embodiment of the present disclosure, the oxidation reaction in step (b) is carried out by mixing the compound of Formula (4) with methyl sulfoxide benzene and benzene, and then adding sodium hydrogen carbonate to subject to a heating reflux reaction to obtain the compound of Formula (5).

The central concept, the technical means employed, and various samples of this application can be fully understood by those of ordinary skill in the art after referring to the following implementation methods.

### BRIEF DESCRIPTION OF THE DRAWINGS

To make the above and other objects, features, advantages and embodiments of this application more apparent and understood, the drawings are described as follows:

FIG. 1 is a flow chart showing the synthesis of a compound of Formula (5) according to an embodiment of the present disclosure. The compound of Formula (5) is one of the precursors for the synthesis of 10′(Z), 13′(E), 15′(E)-Heptadecatrienyl hydroquinone compound (HQ17(3)) represented by Formula (12) used in the present disclosure.

FIG. 2 is a flow chart showing the synthesis of a compound of Formula (10) according to an embodiment of the present disclosure. The compound of Formula (10) is another one of the precursors for the synthesis of 10′(Z), 13′(E), 15′(E)-Heptadecatrienyl hydroquinone compound (HQ17(3)) represented by Formula (12) used in the present disclosure.

FIG. 3 is a flow chart showing the synthesis of 10′(Z), 13′(E), 15′(E)-Heptadecatrienyl hydroquinone compound (HQ17(3)) represented by Formula (12), used in the present disclosure.

FIG. 4 shows the experiment result of the use of 10′(Z), 13′(E), 15′(E)-Heptadecatrienyl hydroquinone compound (HQ17(3)) represented by Formula (12), in inhibiting the replication of the novel coronavirus in cells. In this experiment, the HQ17(13) was firstly dissolved in 0.1% DMSO as stock solution, and then the stock solution was then diluted into different concentrations as 0, 1, 3, 10, 30 uM of HQ17(3) by DMEM medium containing 2% FCS for the experiment.

FIG. 5 shows the quantitative result of the use of 10′(Z), 13′(E), 15′(E)-Heptadecatrienyl hydroquinone compound (HQ17(3)) represented by Formula (12), in inhibiting the replication of the novel coronavirus in cells. In this experiment, the HQ17(13) was firstly dissolved in 0.1% DMSO as stock solution, and then the stock solution was then diluted into different concentrations as 0, 1, 3, 10, 30 uM of HQ17(3) by DMEM medium containing 2% FCS for the experiment.

FIG. 6 shows the experiment result of the use of 10′(Z), 13′(E), 15′(E)-Heptadecatrienyl hydroquinone compound (HQ17(3)) represented by Formula (12), in inhibiting the replication of the novel coronavirus in cells. In this experiment, the HQ17(13) was firstly dissolved in a solvent containing 10% Ethanol and 40% PEG300 as stock solution, and then the stock solution was diluted into different concentrations as 0, 2.5, 5, 7.5, 10, 12.5, 15 uM of HQ17(3) by DMEM medium containing 2% FCS for the experiment.

FIG. 7 shows the quantitative result of the use of 10′(Z), 13′(E), 15′(E)-Heptadecatrienyl hydroquinone compound (HQ17(3)) represented by Formula (12), in inhibiting the replication of the novel coronavirus in cells. In this experiment, the HQ17(13) was firstly dissolved in a solvent containing 10% Ethanol and 40% PEG300 as stock solution, and then the stock solution was diluted into different concentrations as 0, 2.5, 5, 7.5, 10, 12.5, 15 uM of HQ17(3) by DMEM medium containing 2% FCS for the experiment.

### DETAILED DESCRIPTION

To make the description of the present disclosure more detailed and complete, the following illustrative written description of the samples and embodiments of this application are set forth below, but the samples and embodiments of this application are not limited thereto.

Unless otherwise indicated, the scientific and technical proper nouns used herein have the same meaning as commonly understood by those of ordinary skill in the art. Furthermore, the nouns used herein are intended to cover the singular and plural types of the nouns unless otherwise specified.

As used herein, the term “about” generally means that the actual value is within plus or minus 10%, 5%, 1%, or 0.5% of a particular value or range. The term “about” is used herein to mean that the actual value falls within an acceptable standard error scope of the mean, as determined by those of ordinary skill in the art. It should be understood that the scopes, quantities, numerical values, and percentages used herein are modified by the term “about” with the exception of experimental examples, or unless otherwise specified. Therefore, unless otherwise indicated, the numerical values or parameters disclosed in the specification and the appended claims are all approximate values and can be changed according to demand.

This application is characterized by providing a 10′(Z), 13′(E), 15′(E)-Heptadecatrienyl hydroquinone compound (HQ17(3)) represented by Formula (12), and a pharmaceutically acceptable salt, and/or a solvate, and/or a hydrate thereof in preparation of a medicament for treating coronavirus infections and diseases caused by the infections, especially the novel coronavirus (SARS-COV-2) infection.

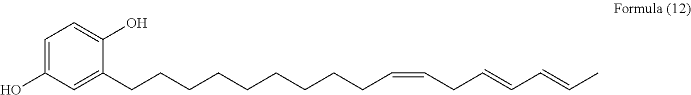 [SMILES: C/C=C/C=C/C/C=C\CCCCCCCCCc1cc(O)ccc1O]

The present disclosure also relates to a pharmaceutical composition, which comprises a therapeutically effective amount of the HQ17(3) compound represented by Formula (12), a pharmaceutically acceptable salt, and/or a solvate, and/or a hydrate, and a pharmaceutically acceptable carrier, auxiliaries or excipient thereof in preparation of a medicament for treating coronavirus infections and diseases caused by the infections, especially the novel coronavirus (SARS-COV-2) infection.

The 10′(Z), 13′(E), 15′(E)-Heptadecatrienyl hydroquinone compound (HQ17(3)) represented by Formula (12), used in the present disclosure is obtained by the chemical synthesis method as mentioned above. The 1, 4-dimethoxybenzene, which is in a stable state since the hydroxyl groups of the hydroquinone are protected by methylation, is used as the starting material. Then, following by subsequent bromoalkylation, demethylation and acetylation protection, iodination and side oxylation, the 2-(10′-oxononyl)-1,4-diacetoxyl benzene is obtained. Then, 2-(10′-oxononyl)-1,4-diacetoxyl benzene and (3E,5Z)-3,5-heptadiene-1-Triphenylphosphine iodide are subjected to undergo the Wittig Reaction and then deacetylation to form 10′(Z), 13′(E), 15′(E)-Heptadecatrienyl hydroquinone compound (HQ17(3)).

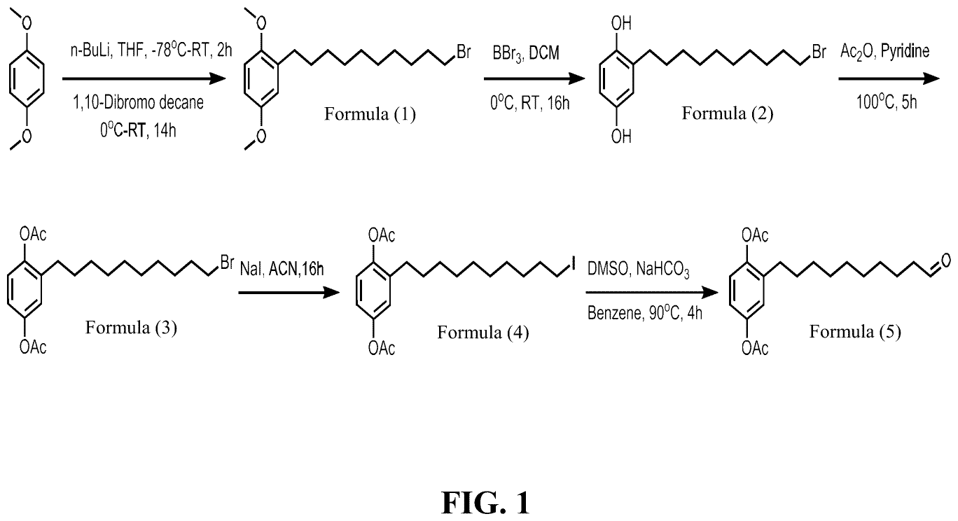

Regarding the method for preparing the compound of Formula (12), please refer to FIG. 1 to FIG. 3 for detailed steps.

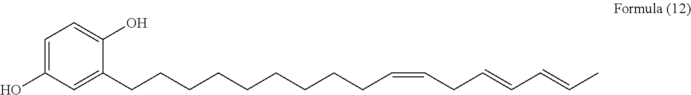 [SMILES: C/C=C/C=C/C/C=C\CCCCCCCCCc1cc(O)ccc1O]

To synthesize the compound of Formula (12), firstly, an intermediate product needs to be synthesized. Please referring to FIG. 1. 1,4-methoxy benzene was mixed with 1, 10-dibromodecane to be alkylated, and demethylated with boron tribromide in an ice bath, and then heated with acetic anhydride under reflux to be acetylated to obtain a compound of Formula (3). Next, an iodination reaction was carried out, and then an oxidation reaction was carried out with DMSO-NaHCO₃ in a benzene solution to obtain a compound of Formula (5).

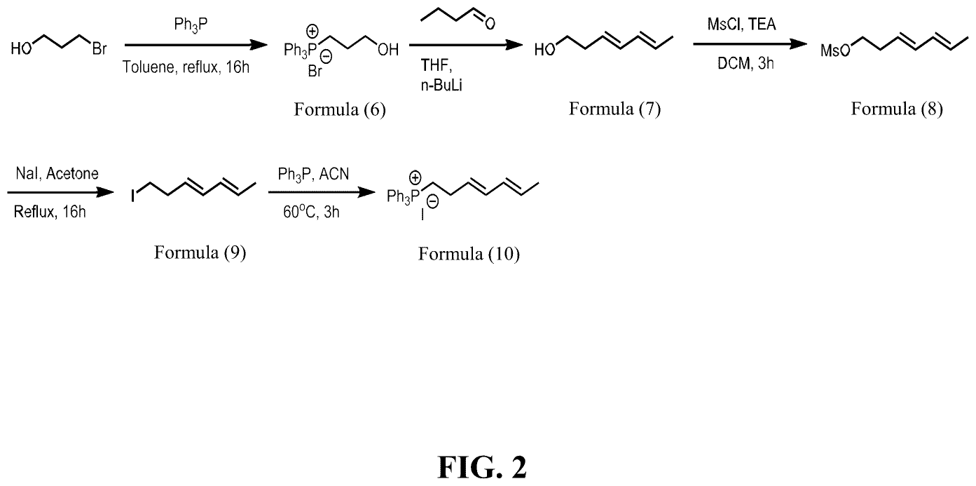

Please referring to FIG. 2. Firstly, 3-bromopropanol as a starting material was reacted with triphenylphosphine to form a dipole body, and then reacted with the dipole body and butadiene aldehyde to generate heptadienol through the Wittig reaction, then heptadienol and methane sulfonyl chloride are subjected to a substitution reaction, and further subjected to an iodination reaction, and finally subjected to a dipolarization reaction with triphenylphosphine to obtain a compound of Formula (10).

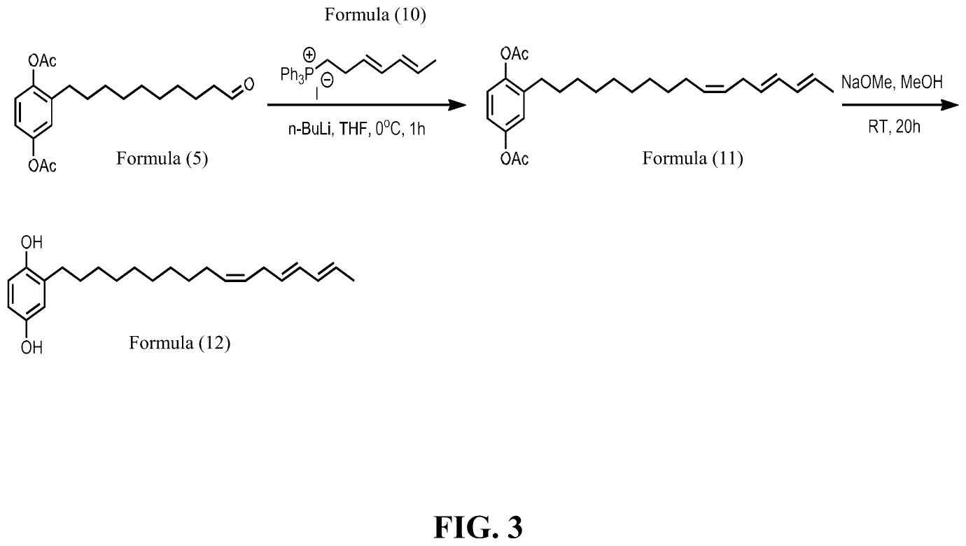

Please referring to FIG. 3, the compound of Formula (5) and the compound of formula (10) (3, 5-heptadien-1-triphenylphosphonium iodide, see FIG. 2 for the synthesis method) were subjected to the Wittig reaction to obtain a compound of Formula (11). The compound of Formula (11) was further reacted with sodium methoxide in methanol and stirred at room temperature overnight to obtain the compound of Formula (12).

Please refer to the following Embodiment 1 for the information with regard to the detailed chemical synthesis method of 10′(Z), 13′(E), 15′(E)-Heptadecatrienyl hydroquinone compound (HQ17(3)) represented by Formula (12), used in this disclosure, and the qualitative and quantitative verification of each related intermediate product thereof.

### Embodiment 1 Method for Preparing Heptadecatrienyl Hydroquinone Compound of Formula (12)

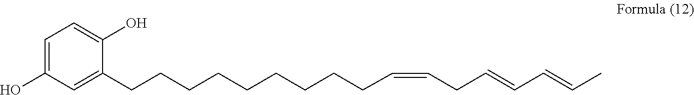 [SMILES: C/C=C/C=C/C/C=C\CCCCCCCCCc1cc(O)ccc1O]

### Embodiment 1.1 Preparation of Compound of Formula (1)

1,4-dimethoxybenzene (10 g, 72.4 mmol) and n-butyllithium (5.8 g, 90.5 mmol) were taken and put in a round-bottom flask, and added with a tetrahydrofuran solvent (200 mL), and then the reaction flask was placed in a low temperature reactor; after stirring was carried out at a temperature of −78° C. for 1 hour, the reaction flask was transferred to room temperature and stirring was carried out for 1 hour; a single-neck flask was additionally prepared, 1,10-dibromodecane (65.17 g, 217.2 mmol) was taken and dissolved in tetrahydrofuran (100 mL), and the solution was slowly dropped into the original solution, and then stirred at room temperature for 14 hours, and then the product was purified by a silica gel column to obtain 17 g of a product, which is 2-(10′-bromodecyl)-1,4-dimethoxybenzene (namely, a compound of Formula (1)), with a yield of 65%.

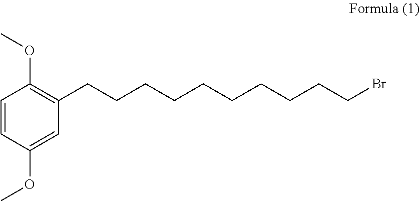 [SMILES: COc1ccc(OC)c(CCCCCCCCCCBr)c1]

¹H NMR (500 MHZ, CDCl₃) δ 6.75-6.76 (d, J=4 Hz, 1H, 6-H), 6.72 (d, J=2.5 Hz, 1H, 3-H), 6.67 (dd, J=9, 2.5 Hz, 1H, 5-H), 3.77 (s, 6H, 1-OCH₃ and 4-OCH₃), 3.40 (t, J=6.5 Hz, 2H, 10′-CH₂), 2.56 (t, J=7.5 Hz, 2H, l′-CH₂), 1.84 (m, 2H, 9′-CH₂), 1.53-1.58 (m, 2H, 2′-CH₂), 1.38-1.43 (m, 2H, 8′-CH₂), 1.28-1.37 (m, 10H, 3′ to 7′-(CH₂) 5).

### Embodiment 1.2 Preparation of Compound of Formula (2)

The compound of Formula (1) (5 g, 14.0 mmol) was taken from a round-bottom flask and dissolved in dichloromethane (300 mL), slowly added drop wise with boron tribromide (4 mL, 42 mmol) in an ice bath, and stirred at room temperature for 16 hours, and then the product was purified by a silica gel column to obtain 3.5 g of a product, which is 2-(10′-bromodecyl)-1,4-dihydrooxybenzene (a compound of formula (2)), with a yield of 76%.

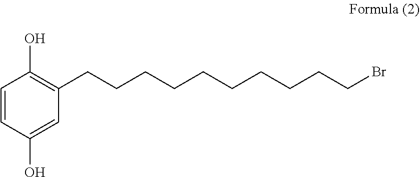 [SMILES: Oc1ccc(O)c(CCCCCCCCCCBr)c1]

¹H NMR (500 MHZ, CDCl₃) δ 6.3-6.65 (d, J=8.5 Hz, 1H, 6-H), 6.62 (d, J=2.5 Hz, 1H, 3-H), 6.53-6.56 (dd, J=8.5, 2.5 Hz, 1H, 5-H), 3.40 (t, J=6.5 Hz, 2H, 10′-CH₂), 2.54 (t, J=7.5 Hz, 2H, 1′-CH₂), 1.85 (m, 2H, 9′-CH₂), 1.56-1.60 (m, 2H, 2′-CH₂), 1.38-1.43 (m, 2H, 8′-CH₂) 1.25-1.33 (m, 10H, 3′ to 7′-(CH₂) 5).

### Embodiment 1.3 Preparation of Compound of Formula (3)

The compound 2 of Formula (2) (16 g, 48.6 mmol) was taken from a round-bottom flask and dissolved in pyridine (100 mL), added with acetic anhydride (7.1 mL, 97.2 mmol) at room temperature, stirred under reflux at a temperature of 110° C. for 5 hours, and then concentrated under reduced pressure, and the product was purified by silica gel chromatography to obtain 13 g of a product, which is 2-(10′-bromodecyl)-1,4-diacetoxyl benzene (namely, a compound of Formula (3)), with a yield of 64%.

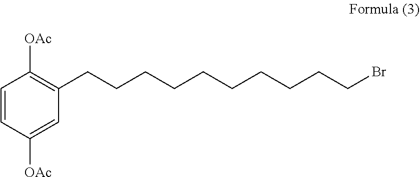 [SMILES: CC(=O)Oc1ccc(OC(C)=O)c(CCCCCCCCCCBr)c1]

¹H NMR (500 MHZ, CDCl₃) δ 7.00-7.02 (d, J=8.5 Hz, 1H, 6-H), 6.96 (d, J=2.5 Hz, 1H, 3-H), 6.91-6.94 (dd, J=8.5, 2.5 Hz, 1H, 5-H), 3.40 (t, J=6.5 Hz, 2H, 10′-CH₂), 2.48 (t, J=7.5 Hz, 2H, l′-CH₂), 2.30 (s, 3H, 1-COCH₃), 2.28 (s, 3H, 4-COCH₃), 1.85 (m, 2H, 9′-CH₂), 1.52-1.55 (m, 2H, 2′-CH₂), 1.39-1.43 (m, 2H, 8′-CH₂) 1.28-1.37 (m, 10H, 3′ to 7′-(CH₂) 5).

### Embodiment 1.4 Preparation of Compound of Formula (4)

The compound of Formula (3) (5.0 g, 12.1 mmol) was taken from a round-bottom flask and dissolved in acetonitrile, added with sodium iodide (2.7 g, 18.2 mmol), and then stirred for 16 hours; after the reaction was finished, the product was purified by a silica gel column to obtain 2-(10′-iododecyl)-1,4-diacetoxyl benzene (4 g) (a compound of Formula (4)), with a yield of 71%.

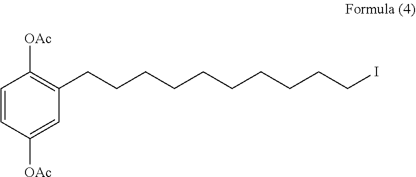 [SMILES: CC(=O)Oc1ccc(OC(C)=O)c(CCCCCCCCCCI)c1]

¹H NMR (500 MHZ, CDCl₃) δ 7.00-7.02 (d, J=8 Hz, 1H, 6-H), 6.96 (d, J=3 Hz, 1H, 3-H), 6.91-6.94 (dd, J=8.5, 2 Hz, 1H, 5-H), 3.18 (t, J=6.5 Hz, 2H, 10′-CH₂), 2.48 (t, J=7.5 Hz, 2H, l′-CH₂), 2.30 (s, 3H, 1-COCH₃), 2.28 (s, 3H, 4-COCH₃), 1.81 (m, 2H, 9′-CH₂), 1.52-1.57 (m, 2H, 2′-CH₂), 1.35-1.39 (m, 2H, 8′-CH₂) 1.28-1.34 (m, 10H, 3′ to 7′-(CH₂)₅).

### Embodiment 1.5 Preparation of Compound of Formula (5)

The compound of Formula (4) (2 g, 4.3 mmol) was taken and put in a round-bottom flask with dimethyl sulfoxide (100 mL) and benzene (100 mL) as solvents, then added with sodium bicarbonate and stirred under reflux at a temperature of 90° C. for 4 hours, and then added with water (100 mL), an organic layer was taken and quenched and washed twice with a 0.1 N hydrochloric acid solution, and then quenched and washed once with a saturated salt solution, the organic layer was taken and concentrated under pressure after water was removed by sodium sulfate, and the product was separated and purified by a silica gel column to obtain 2-(10′-oxononyl)-1,4-diacetoxyl benzene (1.4 g) (a compound of Formula (5)), with a yield of 96%.

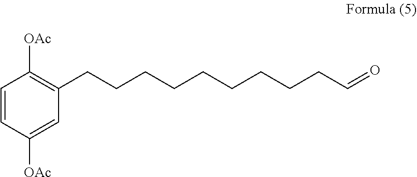 [SMILES: CC(=O)Oc1ccc(OC(C)=O)c(CCCCCCCCCC=O)c1]

¹H NMR (500 MHZ, CDCl₃) δ 9.76 (s, 1H, 10′-H), 7.00-7.02 (d, J=8 Hz, 1H, 6-H), 6.96 (d, J=3 Hz, 1H, 3-H), 6.91-6.94 (dd, J=8.5, 2 Hz, 1H, 5-H), 2.48 (t, J=7.5 Hz, 2H, 9′-CH₂), 2.41 (t, J=7.5 Hz, 2H, 1′-CH₂), 2.31 (s, 3H, 1-COCH₃), 2.28 (s, 3H, 4-COCH₃), 1.60 (m, 2H, 8′-CH₂), 1.51-1.56 (m, 2H, 2′-CH₂), 1.24-1.36 (m, 10H, 3′ to 7′-(CH₂)₅).

### Embodiment 1.6 Preparation of Compound of Formula (6)

3-bromopropanol (55.6 g, 0.4 mol) and triphenylphosphine (150 g, 0.6 mol) were taken and put in a round-bottom flask, and added with toluene (200 mL) to be dissolved, and the solution was stirred under reflex at a temperature of 110° C. for 16 hours; after cooled to room temperature, the solution was added with ethyl ether so that the product was precipitated to obtain (3-hydroxypropyl)triphenylphosphonium bromide (160 g) (a compound of Formula (6)), with a yield of 99%.

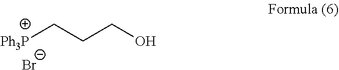

¹H NMR (500 MHZ, DMSO-D6) δ 7.59-7.92 (m, 15H,-(phenyl) 3), 3.52-3.61 (m, 4H, 2, 3-CH₂CH₂), 1.63-1.70 (m, 2H, 1-CH₂).

### Embodiment 1.7 Preparation of Compound of Formula (7)

The compound of Formula (6) (103 g, 0.26 mol) was taken and dissolved in anhydrous tetrahydrofuran (100 mL), added with n-butyl lithium (208 mL, 2.5 M in hexane, 0.52 mol) in a 0° C. ice bath, and continuously stirred for half an hour in a 0° C. ice bath, then added with bis-crotonaldehyde (25.8 mL, 0.31 mol) and continuously stirred for 1 hour, and then added with saturated ammonium chloride (100 mL) to terminate the reaction, the product was extracted twice with ethyl ether (50 mL×2), organic layers were combined, pressure reduction, concentration and draining were performed, and the product was purified by silica gel column chromatography to obtain (3E, 5E)-3, 5-heptadien-1-ol (18.6 g) (a compound of Formula (7)), with a yield of 62%.

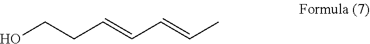

¹H NMR (500 MHz, CDCl₃) δ=1.55-1.78 (d, 3H, 7-CH₃), 2.31-2.47 (dt, 2H, 2-CH₂), 3.67 (m, 2H, 1-CH₂OH), 5.48-5.54 (dt, 1H, 6-═CH—CH₃), 5.63-5.76 (dq, 1H, 3-CH═CH), 6.01-6.34 (m, 2H, 4-═CH—CH and 5-CH═CH).

### Embodiment 1.8 Preparation of Compound of Formula (8)

The compound of Formula (7) (0.5 g, 4.46 mmol) was taken and dissolved in dichloromethane (100 mL), then added with methane sulfonyl chloride (414.1 mL, 5.35 mmol) and triethylamine (932.4 mL, 6.69 mmol) in a 0° C. ice bath, transferred to room temperature and stirred for 3 hours, and added with dichloromethane (100 mL) to dilute the solution, and the solution was quenched and washed twice with 1 M hydrochloric acid (50 mL), and quenched and washed twice with saturated sodium carbonate (50 mL), and finally quenched and washed once with saturated salt (50 mL), then organic layers were combined, and then concentrated under reduced pressure after water was removed with sodium sulfate, without purifying any more in this step, to obtain (3E, 5E)-3, 5-heptadien-1-methylsulfonic acid (630 mg) (a compound of Formula (8)), with a yield of 74%.

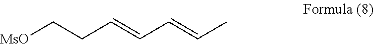

### Embodiment 1.9 Preparation of Compound of Formula (9)

In a round-bottom flask, the compound of Formula (8) (620 mg, 3.26 mmol) was taken and dissolved in acetone (50 mL), and added with sodium iodide (1.46 g, 9.78 mmol) and copper powder (6.9 mg, 0.1 mmol) in a 0° C. ice bath to react under reflux at a temperature of 60° C. for 2 hours; after the reaction was finished, the solution was added with saturated sodium thiosulfate (50 mL) and extracted with dichloromethane (50 mL), and an organic layer was taken and concentrated. The product was purified by a silica gel chromatography column to obtain (2E, 4E)-7-iodo-2, 4-heptadiene (600 mg) (compound of Formula (9)), with a yield of 82%.

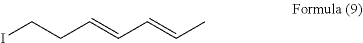

¹H NMR (500 MHZ, CDCl₃) δ=1.72-1.78 (d, 3H, 1-CH₃), 2.60-2.77 (dt, 2H, 6-CH₂), 3.15 (m, 2H, 7-CH₂), 5.43-5.49 (dt, 1H, 2-CH—CH), 5.63-5.78 (dq, 1H, 5-═CH—CH₂), 6.00-6.09 (m, 2H,3-═CH—CH and 4-CH═CH).

### Embodiment 1.10 Preparation of Compound of Formula (10)

The compound of formula (9) (2.2 g, 9.9 mmol) was taken and dissolved in acetonitrile (200 mL), and added with triphenylphosphine (3.9 g, 14.8 mmol) and stirred under reflux at a temperature of 90° C. for 16 hours. The solution was filtered after the stirring was finished, then the solution was concentrated under reduced pressure, and an oily product was washed with ethyl ether and dried to obtain 2.5 g of an oily product, which is (3E, 5E)-3, 5-heptadien-1-triphenylphosphonium iodide (a compound of Formula (10)), with a yield of 70%.

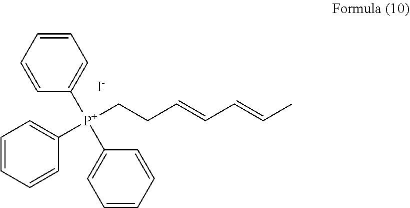 [SMILES: C/C=C/C=C/CC[P+](c1ccccc1)(c1ccccc1)c1ccccc1 ; [I-]]

### Embodiment 1.11 Preparation of Compound of Formula (11)

The compound of Formula (10) (0.6 g, 1.7 mmol) and n-butyllithium (0.16 g, 2.5 mmol) were taken and dissolved in tetrahydrofuran (100 mL) and stirred at room temperature for 30 minutes to form a dipolar body product; in addition, a reaction flask was additionally prepared, the compound 5 (0.59 g, 1.7 mmol) was taken and dissolved in tetrahydrofuran (50 mL), and stirred at a temperature of 0° C., and the dipolar body product was poured into the reaction flask and continuously stirred for 30 minutes, the reaction was terminated with ammonium chloride, the product was extracted with benzene, and concentrated and dried after water was removed with magnesium sulfate. The product was purified by HPLC to obtain 2-(10′Z, 13′E, 15′E)-10′, 13′, 15′-heptadecatrienyl-1,4-p-diacetyl benzene (341 Mg) (a compound of formula (11)), with a yield of 32%.

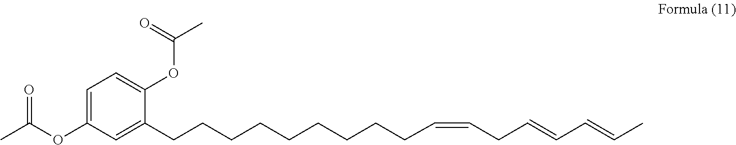 [SMILES: C/C=C/C=C/C/C=C\CCCCCCCCCc1cc(OC(C)=O)ccc1OC(C)=O]

¹H NMR (500 MHZ, CDCl₃) δ=1.26-1.33 (m, 12H, 3′ to 8′-(CH₂) 6), 1.51-1.57 (m, 2H, 2′-CH₂), 1.71-1.78 (d, 3H, 17′-CH₃), 1.97-2.08 (m, 2H, 9′-CH₂), 2.28 (s, 3H, Acetyl-CH₃), 2.31 (s, 3H, Acetyl-CH₃), 2.48 (t, 2H, l′-CH₂), 2.79 (t, 2H, 12′-CH₂), 5.26-5.45 (m, 2H, 10′-CH═ and 11′-═CH), 5.49-5.62 (m, 2H, 13′-CH═ and 16′-═CH), 5.93-6.05 (m, 2H, 14′ to 15′-═CH—CH═), 6.91-6.94 (dd, 1H, 5-ArH), 6.96 (d, 1H, 3-ArH), 7.01 (d, 1H, 6-ArH).

### Embodiment 1.12 Preparation of Compound of Formula (12)

The compound of Formula (11) (22.5 g, 52.7 mmol) was reacted with sodium methoxide (0.6 g, 10.5 mmol) in methanol (300 mL), and stirred at room temperature for 20 hours, then the solution was neutralized with acid resin, the solution was extracted with benzene after being filtered, and finally organic solutions were combined and evaporated to dryness under reduced pressure. The product was purified by high performance liquid chromatography to obtain a solid product 2-(10′Z, 13′E, 15′E)-10′, 13′, 15′-heptadecatrienyl-1, 4-p-diphenol (7.2 g) (a compound of formula (12)), with a yield of 39%.

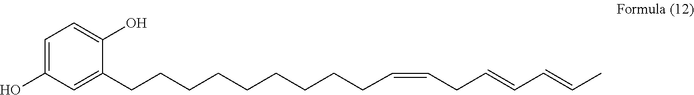 [SMILES: C/C=C/C=C/C/C=C\CCCCCCCCCc1cc(O)ccc1O]

¹H NMR (500 MHZ, CDCl₃) δ=1.27-1.34 (m, 12H, 3′ to 8′-(CH₂) 6), 1.54-1.61 (m, 2H, 2′-CH₂), 1.78-1.71 (d, 3H, 17′-CH₃), 1.96-2.08 (m, 2H, 9′-CH₂), 2.53 (t, 2H, 1′-CH₂), 2.79 (t, 2H, 12′-CH₂), 4.31-4.33 (s, 2H, 1 and 4-ArOH), 5.33-5.45 (m, 2H, 10′-CH═ and 11′-═CH), 5.49-5.62 (m, 2H, 13′-CH═ and 16′-═CH), 5.95-6.05 (m, 2H, 14′ to 15′-═CH—CH═), 6.55 (dd, 1H, 5-ArH), 6.61 (d, 1H, 3-ArH), 6.64 (d, 1H, 6-ArH); ¹³C NMR (500 MHz, CDCl₃) δ=18.15 (C-17′), 27.27 (C-9′), 29.4 (C-2′), 29.58-29.82 (C-3′ to 8′), 30.19 (C-1′), 30.45 (C-12′), 113.15 (ArC-5), 116.1 (ArC-6), 116.93 (ArC-3), 127.08 (C-11′), 127.34 (C-16′), 130.1 (C-13′), 130.19 (ArC-2), 130.59 (C-14′), 131.1 (C-10′), 131.68 (C-15′), 147.49 (ArC-1), 149.48 (ArC-4).

### Embodiment 2: The Use of 10′(Z), 13′(E), 15′(E)-Heptadecatrienyl Hydroquinone Compound (HQ17(3)) Represented by Formula (12), in Inhibiting the Replication of the Novel Coronavirus in Cells, Wherein the HQ17(13) was Firstly Dissolved in 0.1% DMSO as Stock Solution, and then the Stock Solution was Diluted into Different Concentrations as 0, 1, 3, 10, 30 uM of HQ17(3) by DMEM Medium Containing 2% FCS for the Experiment

The HQ17(3) represented by Formula 12, synthesized according to the aforementioned synthesis method was firstly dissolved in 0.1% DMSO at 10 mM concentration as a stock solution. Then the stock solution was diluted into different concentrations as 0, 1, 3, 10, 30 uM of HQ17(3) by DMEM medium containing 2% FCS for the virus inhibition experiment.

The infectivity of the novel coronavirus (SARS-COV-2) was tested by using Vero E6 cells. Virus titers were determined by performing Vero E6 cells plaque assays. The detailed procedure is as follows:

Vero E6 cells (ATCC® CRL-1586™, 2×10⁵ cells/well) were firstly cultured with DMEM medium (containing 10% fetal calf serum and antibiotics) for one day, and then inoculated with the novel coronavirus (SARS-COV-2) (NTU49, GISAID: EPI_ISL_1010728) (50-100 plaque forming unit (PFU)/well) for one hour at 37° C. Next, washed the cells with phosphate solution (PBS) to remove viruses that have not entered the cells.

Covered the cells with 1% methylcellulose (Sigma, cat #M0387) and 0, 1, 3, 10, 30 uM of HQ17(3). After one week, cells were fixed overnight with 10% formaldehyde solution (Marcon™ Chemicals, cat #H121-08). Next, after removing the upper layer of methylcellulose, the cells were stained with crystal violet, and the number of viral plaques was counted. The virus inhibition rate is calculated as [1−(VD/VC)]×100%, where VD is the number of virus plaques in the presence of HQ17(3), and VC is the number of virus plaques in the absence of HQ17(3).

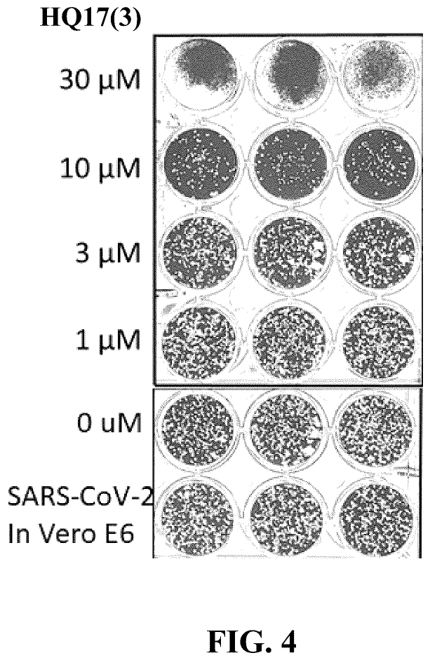

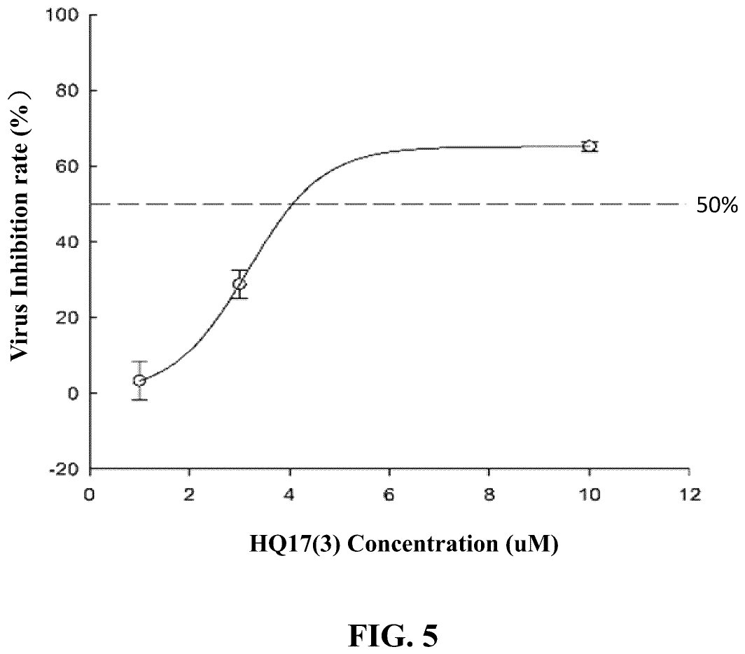

Please refer to FIG. 4 and FIG. 5 for the experiment result. FIG. 4 shows the viral plaque assay experiment result of inhibiting virus replication in Vero E6 cells infected with the novel coronavirus (SARS-COV-2) using different concentrations of HQ17(3) such as 0, 1, 3, 10, and 30 uM. The viral plaque assay experiment result shown in FIG. 4 was then quantified and presented in FIG. 5. The result shown in FIG. 5 indicated that when 10 uM of HQ17(3) was administered to Vero E6 cells infected with the novel coronavirus (SARS-COV-2), the HQ17(3) was able to inhibit the replication of the novel coronavirus (SARS-COV-2) in the cells by more than 60%, comparing with the control group which was not administered with HQ17(3). That is to say, under the conditions of this experiment, 10 uM of HQ17(3) can show a virus inhibition rate of greater than 60%.

### Embodiment 3: The Use of 10′(Z), 13′(E), 15′(E)-Heptadecatrienyl Hydroquinone Compound (HQ17(3)) Represented by Formula (12), in Inhibiting the Novel Coronavirus Replication in the Cells, Wherein the HQ17(13) was Firstly Dissolved in a Solvent Containing 10% Ethanol and 40% PEG300 as Stock Solution, and then the Stock Solution was Diluted into Different Concentrations as 0, 2.5, 5, 7.5, 10, 12.5, 15 uM of HQ17(3) by DMEM Medium Containing 2% FCS for the Experiment

The HQ17(3) represented by Formula 12, synthesized according to the aforementioned synthesis method was firstly dissolved in a solvent containing 10% Ethanol and 40% PEG300 as stock solution. Then the stock solution was diluted into different concentrations as 0, 2.5, 5, 7.5, 10, 12.5, 15 uM of HQ17(3) by DMEM medium containing 2% FCS for the virus inhibition experiment.

The infectivity of the novel coronavirus (SARS-COV-2) was tested by using Vero E6 cells. Virus titers were determined by performing Vero E6 cells plaque assays. The detailed procedure is as follows:

Vero E6 cells (ATCCR CRL-1586™, 2×10⁵ cells/well) were firstly cultured with DMEM medium (containing 10% fetal calf serum and antibiotics) for one day, and then inoculated with the novel coronavirus (SARS-COV-2) (NTU49, GISAID: EPI_ISL_1010728) (50-100 plaque forming unit (PFU)/well) for one hour at 37° C. Next, washed the cells with phosphate solution (PBS) to remove viruses that have not entered the cells.

Covered the cells with 1% methylcellulose (Sigma, cat #M0387) and 0, 2.5, 5, 7.5, 10, 12.5, 15 uM of HQ17(3). After one week, cells were fixed overnight with 10% formaldehyde solution (Marcon™ Chemicals, cat #H121-08). Next, after removing the upper layer of methylcellulose, the cells were stained with crystal violet, and the number of viral plaques was counted. The virus inhibition rate is calculated as [1−(VD/VC)]×100%, where VD is the number of virus plaques in the presence of HQ17(3), and VC is the number of virus plaques in the absence of HQ17(3).

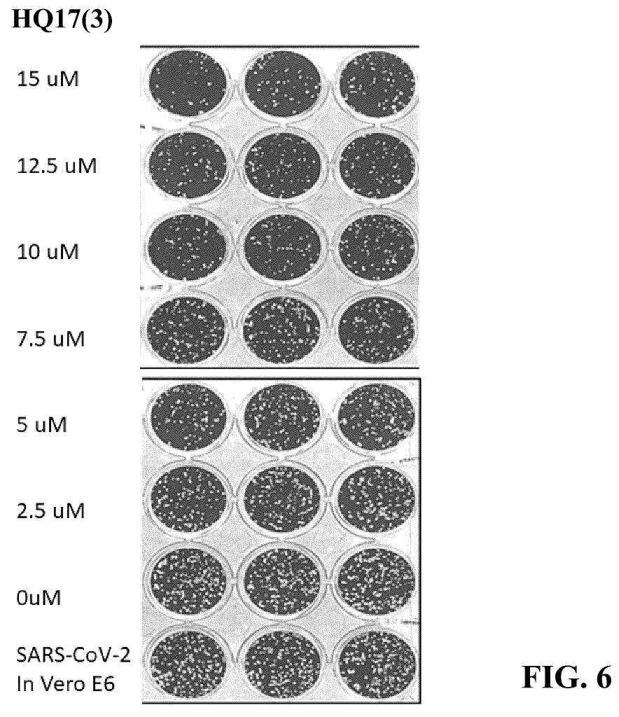

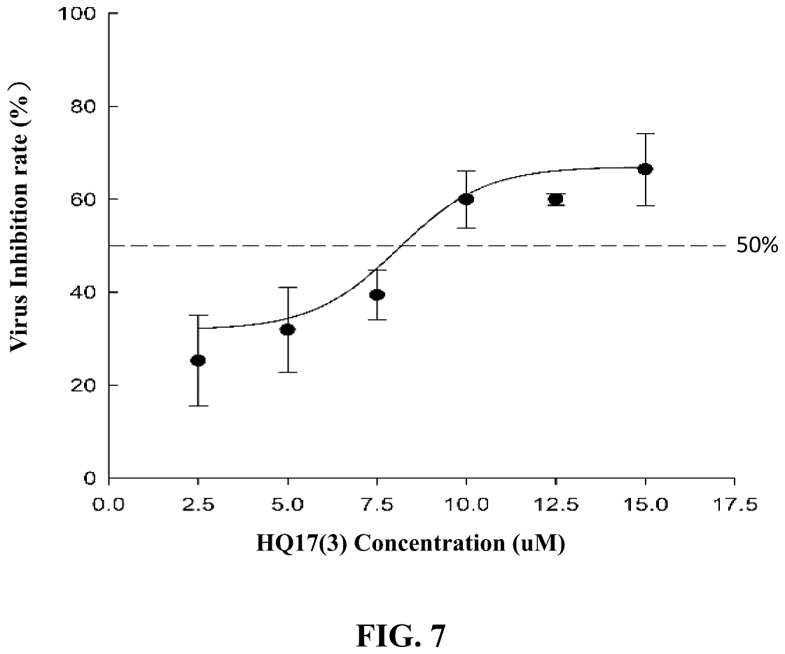

Please refer to FIG. 6 and FIG. 7 for the experiment result. FIG. 6 shows the viral plaque assay experiment result of inhibiting virus replication in Vero E6 cells infected with the novel coronavirus (SARS-COV-2) using different concentrations of HQ17(3) such as 0, 2.5, 5, 7.5, 10, 12.5, 15 uM. The viral plaque assay experiment result shown in FIG. 6 was then quantified and presented in FIG. 7. The result shown in FIG. 7 indicated that when 8.5±0.96 uM of HQ17(3) was administered to Vero E6 cells infected with the novel coronavirus (SARS-COV-2), the HQ17(3) was able to inhibit the replication of the novel coronavirus (SARS-COV-2) in the cells by 50%, comparing with the control group which was not administered with HQ17(3). That is to say, under the conditions of this experiment, 8.5±0.96 uM of HQ17(3) can show a virus inhibition rate of greater than 50% (i.e. EC50=8.5±0.96 uM).

It can be confirmed from the above embodiments that the 10′(Z), 13′(E), 15′(E)-Heptadecatrienyl hydroquinone compound (HQ17(3)) represented by Formula (12) exhibits the ability of inhibiting the viral replication and growth in the cells infected by coronavirus, especially the novel coronavirus (SARS-COV-2). Thus, the HQ17(3) can therefore be used in preparation of a medicament for treating coronavirus infections and the diseases caused by the infections, especially the novel coronavirus (SARS-COV-2) infection.

Although this application has been disclosed above by way of embodiments, it is not intended to limit this application. Various changes and modifications can be made by those of ordinary skill in the art without departing from the spirit and scope of this application. Therefore, the scope of protection of this application is defined by the appended claims.

### SYMBOL DESCRIPTION

None

What is claimed is:

1. A method for treating a coronavirus infection or a disease caused by the coronavirus infection in a subject in need thereof, the method comprising administrating to the subject a therapeutically effective amount of (a) a compound represented by Formula (12), a pharmaceutically acceptable salt, a solvate, or a hydrate thereof, 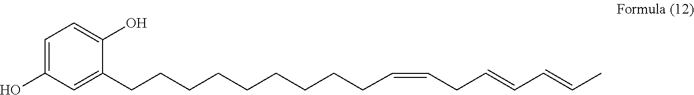 [SMILES: C/C=C/C=C/C/C=C\CCCCCCCCCc1cc(O)ccc1O]
or, (b) a pharmaceutical composition comprising a compound represented by Formula (12), or a pharmaceutically acceptable salt, a solvate, or a hydrate thereof.

2. The method according to claim 1, wherein the coronavirus is HCOV-229E alpha coronavirus, HCoV-NL63 alpha coronavirus, HCoV-HKU1 beta coronavirus, HCoV-OC43 beta coronavirus, severe acute respiratory syndrome coronavirus (SARS-COV), Middle East Respiratory Syndrome coronavirus (MERS-COV) or the novel coronavirus (SARS-COV-2).

3. The method according to claim 2, wherein the coronavirus is SARS-COV-2.

4. The method according to claim 1, wherein the subject in need is a coronavirus-infected mammal.

5. The method according to claim 1, wherein the subject in need is a coronavirus-infected human.

6. The method according to claim 1, wherein the disease caused by the coronavirus infection is a respiratory disease caused by SARS-COV or SARS-COV-2.

7. The method according to claim 1, wherein the pharmaceutical composition comprising a compound represented by Formula (12) further comprising a pharmaceutically acceptable carrier, auxiliaries or excipient.

8. The method according to claim 1, wherein the pharmaceutical composition comprising a compound represented by Formula (12) is a solid preparation, an injection, a spray, a liquid pharmaceutical or a composite pharmaceutical.

9. A method for inhibiting the replication of coronavirus in a mammal in need thereof, the method comprising administrating to the mammal a therapeutically effective amount of (a) a compound represented by Formula (12), a pharmaceutically acceptable salt, a solvate, or a hydrate thereof, 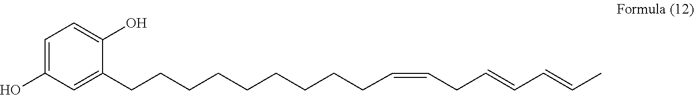 [SMILES: C/C=C/C=C/C/C=C\CCCCCCCCCc1cc(O)ccc1O]
or, (b) a pharmaceutical composition comprising a compound represented by Formula (12), or a pharmaceutically acceptable salt, a solvate, or a hydrate thereof.

10. The method according to claim 9, wherein the mammal in need is a coronavirus-infected human.

11. The method according to claim 9, wherein the mammal in need is a human being infected by SARS-COV or SARS-COV-2.
